(19) United States
(12) Patent Application Publication
(10) Pub. No.: US 2002/0047991 A1
(43) Pub. Date: Apr. 25, 2002

(54) Method for testing visual functions of a human eye and perimeter for carrying out the method

(75) Inventor: Peter Jaggi, Oberrohrdorf (CH)

Correspondence Address: William H. Logsdon, 700 Koppers Building, 436 Seventh Avenue, Pittsburgh, PA, 15219-1818, US

(73) Assignee: INTERZEAG AG

(21) Appl. No.: 10/000,974

(22) Filed: Oct. 24, 2001

(30) Foreign Application Priority Data
Oct. 25, 2000 (EP) 00 810 984.5

(51) Int. Cl. A61B 3/14

(52) U.S. Cl. 351/209

(57) ABSTRACT

A method for testing the visual functions of a human eye by means of a perimeter which is placed at a location allowing observation, by the eye to be tested, along an observation axis. The method makes use of means associated with a light source for the chronologically staggered generation of stimuli at selected locations in the vicinity of a fixation mark located on the observation axis, and makes use of a camera linked to a computer for the observation of the position of the eye and direction of gaze. The method accommodates the diversion of the subject's gaze and incorrect placement of the subject's eye.

### BACKGROUND OF THE INVENTION

1. Field of the Invention

This invention relates to a method for testing the visual functions of a human eye by means of a perimeter which is placed at a location allowing observation, by the eye to be tested, along an observation axis. The method makes use of means associated with a light source for the chronologically staggered generation of stimuli at selected locations in the vicinity of a fixation mark located on the observation axis, and makes use of a camera linked to a computer for the observation of the position of the eye and direction of gaze. The method accommodates the diversion of the subject's gaze and incorrect placement of the subject's eye.

2. Description of the Related Art

Patent document CH-A-677 599 discloses a perimeter for testing the visual functions of a human eye, used in conjunction with a camera with which the position of the subject's eye is measured. If the measurement shows an excessive deviation from the ideal gaze orientation, the test is interrupted, the subject is made aware of the deviation and is repositioned. A plurality of such interruptions can occur during an examination. These interruptions significantly delay the progress of the examination and increase the amount of time required to perform the examination.

A need exists for a method of performing the examination not subject to these interruptions, in which measurements can be carried out in the presence of deviations from the ideal gaze orientation, and in which adjustments to measurements can be made with minimum operator intervention.

### BRIEF DESCRIPTION OF THE INVENTION

Accordingly, the present invention provides a method for testing the visual functions of the human eye, in which compensation is made for mispositioning of the eye to be tested and for diversion of the direction of gaze of the subject. The eye to be tested is located at an observer position. A fixation mark, visible from the observer position, is provided, and the subject is told to direct the gaze of the eye at the fixation mark. A visual stimulus is generated in the vicinity of the fixation mark. During the generation of the visual stimulus, the direction of gaze of the eye is determined, and the deviation of the direction of gaze from the fixation mark is determined. The visual response of the eye to the stimulus is observed. If a deviation of the direction of gaze from the fixation mark is present, the location of the visual stimulus is corrected so that it is placed at the desired location in relation to the direction of gaze. In this way, the desired relationship between the direction of gaze and the location of the visual stimulus can be obtained, regardless of deviation of the direction of gaze from the fixation mark.

### BRIEF DESCRIPTION OF THE DRAWINGS

FIG. 1 is a schematic representation of a perimeter for the performance of the method;

FIG. 2 is a schematic representation of a field of view with an illustration of a scotoma;

FIG. 3 is a schematic representation of an eye position that differs from the ideal position; and

FIG. 4 is a schematic representation of the correction of a stimulus test site.

### DETAILED DESCRIPTION OF THE INVENTION

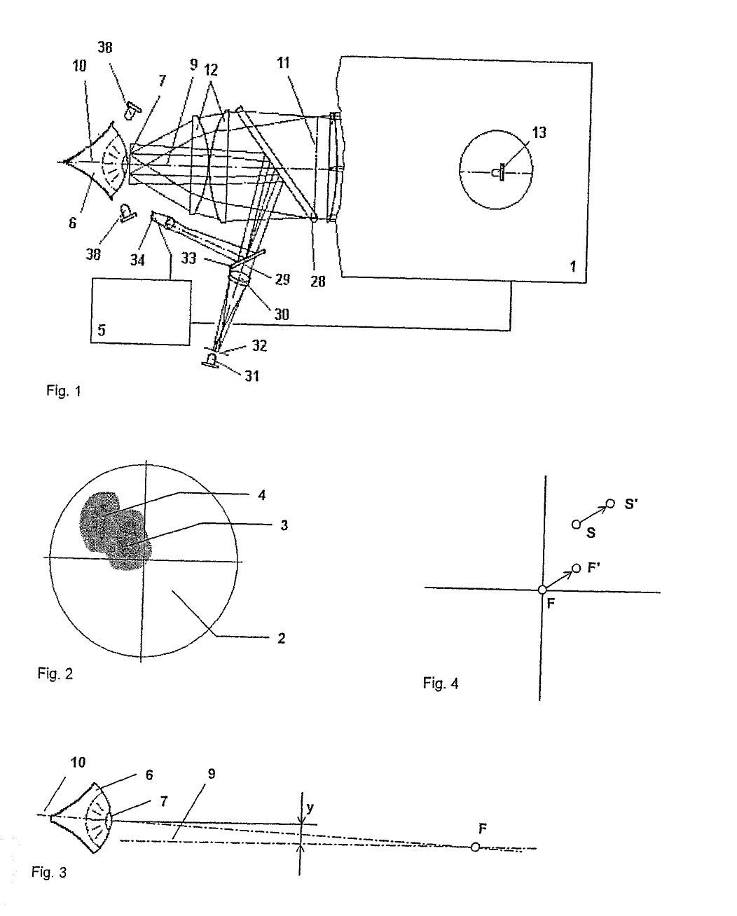

FIG. 1 shows a perimeter 1 of the present invention, housing one or more devices or sources for the generation of visual stimuli.

FIG. 2 shows the results of the determination of the location, in the subject's field of vision 2, of a scotoma. The correct position of the scotoma 3 is shown on the field of vision. An incorrect position of the scotoma 4 may result from an examination, if an eye 6 of the subject is not at the proper location, or is not properly directed.

Referring again to FIG. 1, the perimeter 1 is linked to a computer 5 for control purposes. The eye 6, with pupil 7, of the observer or subject to be examined is located at a defined observer location for an observation. An observation axis 9 extends from the eye 6 to be examined at an observer or subject location 10 to the perimeter. Arranged along observation axis 9 are an intermediate plate 11, located between perimeter 1 and eye 6, and an eyepiece 12, located between intermediate plate 11 and eye 6. Perimeter 1 houses a light source 13 for the generation of visual stimuli at various positions on intermediate plate 11. A partly light-permeable tilted mirror 28 is located between intermediate plate 11 and eyepiece 12. The portion of observation axis 9 between eye 6 and mirror 28 generates, when reflected in mirror 28, an optical axis 29. Lenses 30 are located on axis 29 and have axis 29 as their optical axis. A light source 31 is located on axis 29 on the opposite side of lenses 30 from mirror 28. A diaphragm 32 defines the shape and size of a fixation mark on axis 9 as an aid to the orientation of eye 6 of the observer. An additional tilting mirror 33 is located on optical axis 29, between lenses 30 and mirror 28. Mirror 33 is permeable for visible light and reflective for infrared light. An infrared-sensitive charge-coupled device (IR-sensitive CCD) camera 34 is placed so that its field of view extends along axis 29 via mirror 33 and along axis 9 via mirror 28 to eye 6. Infrared light-emitting diodes 39 are arrayed near eye 6 to provided illumination for camera 34.

During an examination using suitable means, chronologically staggered visual stimuli are presented to eye 6 from perimeter 1 in the vicinity of observation axis 9. Generation of visual stimuli from perimeter 1 is controlled by means of a computer 5. To expedite orienting the subject's eye 6 with reference to the observation axis 9, the subject is presented with a brightly-lighted mark (fixation mark) on axis 9 as an aid to orientation. Light emitted by light source 31 passes through diaphragm 32, which defines the size and shape of the fixation mark. The light then passes through axis 29, through lenses 30 and mirror 33, to mirror 28, where it is reflected and directed along axis 9 to eye 6.

Eye 6 is observed during the examination by camera 34. For this purpose, eye 6 is illuminated with IR LEDs that are invisible to the subject. The image recorded by camera 34 is evaluated by computer 5. During this evaluation, the position of pupil 7 of eye 6 is determined and the deviations from the correct direction of view or position are evaluated.

FIG. 3 depicts a potential deviation in the direction of view of the subject. The pupil 7 of the eye 6 is in this case located above the optical axis 9. Even if the eye 6 is in this case correctly fixed on the fixation mark, a scotoma would be found in an incorrect position. To correct this deviation, the position of the stimulus test sites is then corrected by calculated values, for example by the value y as illustrated in FIG. 3. The correction is made as necessary for each stimulus and in made in the x and y directions. If the direction of the subject's gaze changes several times during an examination, the position of the stimulus to be presented is corrected a corresponding number of times.

FIG. 4 is a schematic illustration of the correction of the position of a stimulus S. The fixation mark F is located at the zero point of the coordinate system. If the eye 6 is correctly positioned and the direction of the subject's gaze is exactly along the optical axis 9, the camera 34 does not detect any deviation, and a stimulus S, the position of which has not been corrected, is offered. If there is a deviation of the direction of the subject's gaze, for example fixing on the point F′ as illustrated in FIG. 4, this deviation is detected by the camera 34 and converted by computer 5 into a correction coefficient. On the basis of this value, a stimulus S′ is presented at a correspondingly corrected location. The stimulus S′ is thus presented at the correct point relative to the subject's field of view in spite of the deviation in the viewer's gaze.

Having described the currently preferred embodiment of the present invention, it is to be understood that the invention may be otherwise embodied within the scope of the appended claims.

What is claimed is:

1. Method for testing visual functions of a human eye, comprising:
a. locating the eye at an observer position;
b. providing a fixation mark;
c. generating a visual stimulus in the vicinity of the fixation mark;
d. determining the direction of gaze of the eye;
e. determining the deviation of the direction of gaze from the fixation mark;
f. observing the visual response of the eye to the stimulus;
g. correcting the observation of the visual response to the stimulus on the basis of the deviation of the direction of gaze.

2. The method of claim 1, additionally comprising providing a perimeter from which the fixation mark is provided and the visual stimuli are generated.

3. The method of claim 1, additionally comprising a light source by which the visual stimuli are generated.

4. The method of claim 1, wherein the visual stimuli are generated in a chronologically staggered fashion.

5. The method of claim 1, wherein the sequence of steps (b) through (g) is repeated.

6. The method of claim 1, wherein a camera is used to determine the direction of gaze of the eye.

7. The method of claim 6, wherein the camera is an IR-sensitive camera, and wherein the eye is illuminated with infrared emissions.

8. The method of claim 1, wherein a computer is linked to the camera, and wherein the computer converts the deviation of the direction of gaze from the fixation mark to a correction coefficient.

9. The method of claim 1, additionally comprising converting the deviation of the direction of gaze from the fixation mark to a correction coefficient.
